(19) United States
(12) Patent Application Publication
(10) Pub. No.: US 2018/0110725 A1
(43) Pub. Date: Apr. 26, 2018

(54) COMPOSITIONS, METHODS AND KITS FOR THE SAFE INHALED DELIVERY OF TARGETED OPIOIDS FOR THE TREATMENT OF PAIN AND ADDICTION

(71) Applicant: Somniferum Labs LLC, Las Vegas, NV (US)

(72) Inventor: David Lanzkowsky, Las Vegas, NV (US)

(21) Appl. No.: 15/789,856

(22) Filed: Oct. 20, 2017

Related U.S. Application Data
(60) Provisional application No. 62/411,455, filed on Oct. 21, 2016.

(51) Int. Cl. A61K 9/00; A61K 47/10; A61K 31/485; A61K 31/706; A61K 31/675; A61K 47/26; A61M 15/00

(52) U.S. Cl. CPC A61K 9/007; A61K 47/10; A61K 31/485; A61K 31/706; A61K 31/675; A61M 11/041; A61M 15/0086; A61M 2230/04; A61M 2230/20; A61M 2205/36; A61M 2205/6054; A61K 47/26

(57) ABSTRACT

A composition having one or more targeted opioids for administration by inhalation is provided. Targeted opioids include morphine, morphine sulfate, 6 mono acetyl morphine, morphine-6-glucuronide, morphine-6-glucuronide bromide, morphine-6-glucuronide acetate, morphine-6-glucuronide sulfate or other salt forms of these aforementioned substances. One or more targeted opioids is combined with an excipient to formulate a composition useful in the treatment of pain, anxiety or other indications. Herein, these mixtures will be referred to as “opioid compositions.” Compositions are administered in a gaseous state through the use of a novel heat-activated drug inhalation device. Kits and methods of administration of the targeted opioid composition are also provided. The composition and kit are designed for the safe delivery of opioids whereby overdose and death is extremely unlikely because of the properties of the new drug and new delivery system.

### BACKGROUND RATIONALE FOR THE INVENTION

Opioids are a class of analgesic compounds used therapeutically for effective relief of both acute and chronic pain (Fields, Neuron, 2011, 69(4), 591-594). Opioids bind with the μ G-protein coupled receptor located on the membrane of cells in the brain, spinal cord, and gut. Upon binding, opioids act as an agonist, activating the receptor and downstream signaling pathways. Opioids are known for alleviating anxiety, inducing mild sedation, and a sense of “well-being.”

Therapeutic doses can be quite variable from patient to patient, based on the effect of the opioids within each individual patient. Typically, exact dosing regimens are not listed on prescription labels, for example, Oxycontin® tablets range from 10 mg to 160 mg, yet the “Indications and Usage” section states that “[p]hysicians should individualize treatment in every case.”

Despite their therapeutic efficacy, opioids can induce physical chemical dependency, behavioral dependency, and tolerance. Opioids have been associated with a modest (<8%) incidence of iatrogenic (physician-induced) addiction (Volkow and McLellan, N Engl J Med., 2016 Mar. 31; 374(13): 1253-63.). Additionally, opioids are at times abused (2%-26%) following standard medical pain management (Fields, 2011; Volkow and McLellan, 2016). FDA-approved opioids are considered drugs of high abuse potential and are listed under the Controlled Substances Act as Schedule II. Heroin, an opioid of particular concern due to its frequent abuse, addictive potential, overdose risk, and social consequences, cannot be prescribed in the United States and is listed as a Schedule I drug.

More recently, use of FDA-approved opioids is of heightened concern in the United States due to the quadrupling of the opioids overdose death rate over the past 15 years (Volkow and McLellan, 2016). Serious side-effects of opioids, typically administered intravenously (IV) or orally (PO), are respiratory depression and hypotension. Abuse or misuse of opioids using these routes of administration have potential for accidental overdose, magnifying the consequences of the side-effects, and can result in death.

As a result of the growing opioid epidemic, the United States public health officials have initiated a concerted effort to reduce opioid-related deaths. Recently, the FDA released an Opioids Action Plan (Califf et al, N Engl J Med 2016; 374:1480-1485) and the Centers for Disease Control and Prevention (CDC) published a Guideline for Prescribing Opioids for Chronic Pain (Dowell and Chou, MMWR Recomm Rep 2016; 65 (No. RR-1): 1-49). In order to begin to tackle this issue, a safer paradigm for prescribing opioids and treating chronic pain is needed. Further, new approaches to safely treat patients with physical chemical and behavioral dependency, and tolerance are needed.

The present invention is directed toward addressing one or more of the problems discussed above, while prioritizing the patient's health, safety, choice of treatment, reduced adverse effects, and general best interests. An optimized treatment plan will have the added benefits of improvements in social and legal issues for the patient.

### SUMMARY OF THE INVENTION

Compositions

Embodiments herein are directed toward novel opioid compositions, kits and methods that include targeted opioids for novel inhaled administration. For purposes herein a targeted opioid is one or more of morphine, morphine sulfate (both FDA-approved analgesics for which planned Investigational New Drug [IND] applications will be submitted for novel compositions described herein), 6 mono acetyl morphine, synthetic opioids (meperidine, fentanyl, methadone, etc.), semi-synthetic opioids (oxymorphone, hydrocodone, oxycodone, buprenorphine, etc.), morphine-6-glucuronide, morphine-6-bromide, morphine-6-acetate, morphine-6-phosphate, and morphine-6-sulfate or other salt forms of these aforementioned substances (not currently FDA-approved, but planned IND applications to be submitted for the novel compositions described herein). In other embodiments herein, the targeted opioid is further limited to one or more of morphine-6-X, wherein X is PO₄, SO₄, Glucuronide, Acetate, Bromide, or other useful salt form.

Each opioid composition comprises a solution of one or more of the targeted opioids, and one or more select excipients. Select excipients may include propylene glycol, glycerin, polysorbate and/or sucrose solutions. Additionally, pH-adjusting ingredients may be added and include: sodium chloride, sodium hydroxide, and hydrochloric acid. Buffering agents which are “generally regarded as safe” (GRAS) may also be added to ensure an effective and safe pH for the opioid composition. All targeted opioids are soluble in the excipients listed. The opioid composition can include one or more therapeutically active concentrations of the targeted opioid. The varying concentrations will allow for a wide range of active doses. In one aspect of the above composition, the therapeutically active dose used in a single dosing event of a targeted opioid is from about 0.01 to about 15 mg for inhaled administration of heat-activated opioid composition every 3 to 4 hours. In another therapeutically active dose, a single dosing event of a targeted opioid is from about 0.02 to about 10 mg for inhaled administration of heat-activated opioid composition every 3 to 4 hours. Multiple inhalations per dosing event may be required to reach an active dose. These single dosing events may also be every 4-6 hours, every 6-8 hours or 8-12 hours.

In other aspects, the compositions herein can include other non-active ingredients. For example, those that enhance the taste or smell of the composition, like chocolate, rosewater, cinnamon, or other flavorants that are generally regarded as safe (GRAS).

Kits

In another embodiment, a kit having one or more therapeutic doses of a targeted opioid, an inert solution, and a novel heat-activated drug delivery device are provided. In aspects of the kit, instructions on how to use and load the device are also provided.

In some aspects of the kit or medical inhaled drug administration device, the targeted opioid is soluble in an excipient and packaged in secure containers, for example ampoules. Ampoules can be transparent amber containers. Amber-colored glass containers are planned in order to improve stability of the targeted opioid in solution. The opioid composition can be packaged in volumes useful for the novel heat-activated drug inhalation device. For example, the device stores a volume of fluid intended for 10 doses (one per activation of the device). Further, the kit may include morphine-6-glucuronide, for example, dissolved in an excipient in a solution with a volume and concentration combination equal to 10 doses, while accounting for a percentage of the composition which is expected to escape in the ambient air. In some embodiments, the excipient is propylene glycol. These opioid compositions are proposed to be referred to as, for example, morphine-glycol, morphine sulfate-glycol, 6 mono acetyl morphine-glycol, morphine-6-glucuronide-glycol, and morphine-6-glucuronide bromide-glycol, and the like. FDA approval will ultimately determine the names. Moreover, these opioid compositions packaged in an ampule or cartridge are proposed to be referred to as morphine-6-sulfate-glycol cartridges, for example. The ampoule or cartridges are fitted with RFID (Radiofrequency Identification) tags to help monitor inventory. RFID can also be used to insure that the drug is used by a specified date, this will prevent opioid dependent patients from hoarding drugs. That the drug be used by the intended device and the intended user, will mitigate against sharing with un-intended users and devices.

In other aspects, the kit or medical inhaled drug administration device can include one or more doses of naloxone hydrochloride as a safety component. Where the kit includes naloxone hydrochloride, it may also include sterile syringes and/or a nasal dispensing device. The naloxone hydrochloride may also be delivered via inhalation through the same device as already present in the kit or novel heat-activated drug inhalation device. Naloxone hydrochloride is an FDA-approved opioid antagonist indicated for opioid overdose. In emergency situations, this opioid antagonist relieves respiratory depression and/or hypotension. Alternatively the ampoule may contain a double chambered wall with narcan/naltrexone surrounding the active opioid ingredient that would inactivate the opioid upon attempted tampering.

Methods

In yet another embodiment, a component of the novel heat-activated drug inhalation device and method for ensuring the patient is not suffering from low oxygen saturation in the blood before administering a targeted opioid to a patient is provided. The method includes measuring the oxygen saturation of the patient by pulse oximetry. Pulse oximetry involves transmission of 2 different wavelengths of light (typically a red light and an infrared light) through a finger and measuring the resulting wavelengths (the light not absorbed) with a photodetector. While adjusting for ambient light, the 2 resulting wavelengths are used to calculate the percent oxygen saturation. This test ensures the patient in need of the targeted opioid has an acceptable blood oxygen level. One aspect of the method is an acceptable blood oxygen saturation level of 94% or greater.

The heating component of the embodiment for activating an opioid composition of the novel drug inhalation device will be set at a temperature of from about 50° C. to about 500° C. More typically, the temperatures are expected to range from about 50° C. to about 260° C. in the herein described heating component of the medical device. The heating aspect of the device will heat the selected opioid composition so that it changes into a gaseous state. The medical device will include a component which allows for inhalation of the heat-activated opioid composition by the patient in a manner which provides a consistent volume of heat-activated opioid composition for each dose.

In one alternative aspect of the novel heat-activated drug inhalation device and method, the patient has his or her identity confirmed prior to testing his or her blood oxygen level. Identity can be confirmed in any number of ways, including biometric methods such as fingerprint, iris scan, voice analysis, retinal scan, or alternatively including a privately held lock device. The lock device may include a keyfob, a manual or electronic key code. In addition, this alternative aspect will also include the measuring of the patient's oxygen saturation as described above and throughout.

Methods and aspects of the novel drug inhalation device described herein may also include a step of regulating the number of times the subject can receive a dose of the heat-activated targeted opioid composition (thermal vaporization) over any predefined period of time. For example, a subject may be limited to administration of one inhalation of thermally vaporized opioid composition per 1-hour period, per 4-hour period, per 24-hour period, or other length of time as specified by the prescribing physician.

Other features and advantages of the disclosure will become apparent from the following detailed description and claims. Note also that the detailed description is given by way of example, since various changes and modifications within the spirit and scope of the disclosure will be apparent to those skilled in the art.

### DESCRIPTION

Embodiments herein include compositions, kits and methods for inhaled administration of an opioid composition to a patient in need thereof. For purposes herein, any one or more of morphine, morphine sulfate, 6 monoacetylmorphine, synthetic opioids, semi-synthetic opioids, and morphine-6-X, wherein X is PO₄, SO₄, Glucuronide, Acetate, Bromide, or other salt forms, of these aforementioned substances can be referred to as a targeted opioid. As such, where the term “targeted opioid” is used, it refers to at least one of, or a combination of any two of, morphine, morphine sulfate, 6 monoacetylmorphine, synthetic opioids, semi-synthetic opioids, and morphine-6-X, wherein X is PO₄, SO₄, Glucuronide, Acetate, Bromide, or other salt forms of these aforementioned substances. In addition, embodiments herein also refer to compositions that only include combinations of any one or more of, morphine-6-X, wherein X is PO₄, SO₄, Glucuronide, Acetate, Bromide, or other salt forms.

Morphine-6-glucuronide is one of two main metabolites of morphine. Morphine-6-glucuronide is the metabolite which is known to produce the analgesic effect, along with intact morphine, after morphine dosing. The mixture of the morphine-6-glucuronide and the carrier propylene glycol (PG) or vegetable glycerin (VG) or any combination or ratio that may include flavorants that are GRAS (Generally Regarded as Safe), or any ratio of PG/VG mixed with the morphine-6-glucuronide is a new drug composition, which the inventors have named Somnivape™.

For purposes herein, heat-activated (or alternatively, thermal vaporization) refers to a physical change from a liquid state of the novel composition to a gaseous state, produced by heating the liquid solution of the opioid composition to a temperature at which it transitions to a gaseous state.

The administration of an opioid composition is metered at precise dosages allowing only a specific volume of the composition to be administered. Further, the temperature of the heating element will be controlled such that the constituents of the novel opioid compositions will not be degraded, nor allow derivatives to be formed by the high temperature used for heat-activation. Typical heat-activation temperatures herein are from about 50° C. to about 500° C., and more typically from about 50° to about 260° C. In some aspects, the heat-activation temperature is from about 50° C. to about 200° C. Note that the heat-activation temperature can, however, include any temperature required to produce the physical change in the composition from a liquid state to a gaseous state.

In typical embodiments herein, the targeted opioid is combined with an excipient, like propylene glycol, vegetable glycerin, or a mixture of propylene glycol and vegetable glycerin, such that the excipient provides a lower heat-activation temperature, and acts as a carrier of the opioid. In this manner, the excipient allows the targeted opioid to be heat-activated at a temperature lower than needed to heat-activate the opioid alone. The excipient and/or other ingredients also act as a solvent to solubilize and stabilize the targeted opioid. In some embodiments, the targeted opioid is partly solubilized, and in other embodiments, the targeted opioid is fully solubilized in the excipient at ambient temperatures and in the pressure produced in the ampoule or other container.

Excipients herein are typically inert and non-reactive compounds. Illustrative excipients include propylene glycol, glycerin, sucrose, and/or polysorbate solutions. In one aspect the polysorbate is polysorbate 20, polysorbate 40, polysorbate 60, polysorbate 80, or a combination thereof. In another aspect, the glycerin is vegetable glycerin. In other aspects, the excipient is one of either the propylene glycol, glycerin, sucrose, and/or polysorbate solutions. In other aspects, the excipient is a combination of two or more of propylene glycol, glycerin, sucrose, and/or polysorbate solutions. Finally, aspects of the disclosure include a combination of all 4 excipients: propylene glycol, glycerin, sucrose, and/or polysorbate solutions, with the targeted opioid. Note that other excipients are also contemplated to be useful herein, and any excipient that is inert and non-reactive and that lowers the opioid's heat-activation temperature can be used.

The opioid compositions, kits, and methods herein provide currently unrealized benefits. For example, many of the side-effects inherent in an oral or injectable opioid are expected to be minimal with embodiments that utilize inhaled opioid compositions, and more typically, inhaled morphine-6-X, wherein X is PO₄, SO₄, Glucuronide, Acetate, Bromide, or other salt forms as the active ingredient (referred to in the remainder of the disclosure as morphine-6-X compounds or individually as a morphine-6-X compound). The side effects expected to be avoided include drug tolerance, dependence, withdrawal symptoms, respiratory depression, gastro-intestinal distress, constipation, nausea, vomiting, itching, dry mouth, allergy risk, anxiety, depression, appetite suppression, convulsions, hallucinations, miosis, sedation, neuroprotection, dysphoria, stress, post-operative nausea and vomiting, peptic ulcers, cardiac harm (such as stroke), and the like. (Wittwer and Kern, AAPS Journal, 2006; 8 (2) Article 39). Compositions containing one or more of the morphine-6-X compounds, and administered via embodiments herein, can be safely used in renal patients, provided that the dose is appropriately reduced to account for reduced clearance, for example. In addition, inhalation of the morphine-6-X compounds delivers the drug through the cardio-pulmonary tract. The cardio-pulmonary route of drug distribution is typically much quicker and more efficient delivery system than conventional methods. This is particularly relevant for pain medications, where fast and efficient delivery results in removal of discomfort faster, and realizes a reduction in “loading up” on the pain medication while the user is waiting for the drug's effects. These combined proposed effects of the inhaled morphine-6-X compounds, via pulmonary absorption, provides an unexpected improvement in delivery and effect over other conventional oral or injectable opioids. Pulmonary absorption bypasses the liver and reduces the production of harmful opioid metabolites produced by the liver.

Multiple indications for opioid composition usage in a novel heat-activated inhalation drug delivery device are claimed herein. Treatments using the opioid compositions in the pediatric population, or incapacitated adult population, requires an opioid composition neonatal or other opioid composition drug delivery tent. In general, the inhalation of morphine-6-X compounds can be used to treat the following illustrative conditions:
a. Treatment of opioid addiction (in adults and children/babies).
b. Treatment of heroin addiction (in adults and children/babies).
c. Treatment of major depressive disorder and acute depression episodes.
d. Treatment of anxiety, including all subtypes of anxiety (for example PTSD, social anxiety, general anxiety disorder, obsessive compulsive disorder, etc.).
e. Treatment of insomnia (including falling asleep, staying asleep and not waking early).
f. Treatment of acute pain.
g. Treatment of pain (including battlefield injuries, arthritis, and back, replacing epidurals or spinal fusion surgery).
h. Treatment of neuropathic pain states (such as complex regional pain syndrome).
i. Use as a pre-operative sedative in adults in children to treat pain associated with the operation or procedure.
j. Use as a post-operative anesthetic for pain.
k. Use as a general anesthetic.
l. Treatment for aggressive behaviors or in persons at high risk for developing aggressive behaviors.

Considering the aforementioned indications, addition of alkaloids such as caffeine, chocolate and nicotine can be added to the opioid compositions as needed.

Thermal vaporization of the morphine-6-X compound compositions is expected to avoid damage to the compound as induced by combustion, because the temperature of activation is substantially lower than for the solid morphine-6-X compounds. For example, where morphine-6-glucuronide is ignited and smoked, the combustion will also result in the production of toxic compounds formed by the combustion process.

In alterntive embodiments, the target opioid compositions herein can be administered through inhalation through the use of inhalers, squeeze bottles for oral and intranasal inhalation, and gas driven spray atomizers. Further, the target opioid compositions can be administered by inhalation as a solution, suspension, aqueous solution, drops, irrigations, nebulized solution wherein solvent included water and all buffers, dry powder where in dry powder carrier includes lactose and other carriers, single-dose dry powder units, liquid soft mist wherein vehicle included water and other buffers, propellant-based solutions and suspensions wherein propellant included all hydrofluoroalkanes, mucoadhesive solutions, and nasal rinses using devices such as pressurized meter dose inhalers, dry powder inhalers, breath-actuated dry powder inhalers soft mist inhalers, jet nebulizers, ultrasonic nebulizers, vibrating mesh nebulizers, nasal spray bottles, intelligent inhalers, neti pots, intranasal mucosal atomization devices with or without syringes, intranasal vapor of insufflators, spacers used with metered dose inhalers, thermal vaporization aerosol devices.

Another advantage of the composition-inhalation route of administration described herein includes a reduced risk of overdose when used appropriately. An oral dose of an opioid can be subject to overdose because it is easy to take an unsafe dose before the effects of the opioid are realized. Similarly, an intravenous dose can be subject to overdose in the same manner. A transdermal delivery system may also result in overdose and death. However, an inhaled dose has an almost instant effect on the user, and the dose amount is limited by each inhalation. Further, the mental requirements to operate the medical inhalation device would limit a user who has already had a large, but not overly concerning dose. The timing between each inhalation allows time for the effects of the drug to be realized. This time would add to the limitation of mental requirements to operate the medical device.

In addition, at least the morphine-6-X compounds provide a number of benefits over more conventional opioids in the area of opioid dependence and withdrawal. (Wittwer and Kern, 2006). It is disclosed herein that these morphine-6-X compounds have a significantly lower capacity to develop dependence in a user than other conventional opioid or opioid-based drugs. The morphine-6-X compounds appear to act through receptor interactions in the brain that in fact limit drug dependence and addiction, particularly as compared to other conventional opioids.

Without being limited by a particular mechanism, thermally vaporized morphine-6-X compounds, as delivered via the cardio-pulmonary tract, are passed through the Blood Brain Barrier (BBB) to μ receptors found in the brain. The morphine-6-X compounds, once in the brain, spend more time on the receptors as compared to codeine and other like opioids. The use of morphine-6-X compounds avoids the presence of morphine-3-glucuronide, the non-analgesic derivative of morphine. Presence of morphine-3-glucuronide appears to modify morphine-6-X compound activity, allowing the two metabolites to compete for the same μ receptors, or compete for the overall effect of the combined presence of the two drugs.

As discussed, morphine-6-glucuronide is a morphine metabolite having potent analgesic properties. (Wittwer and Kern, 2006). Morphine-6-glucuronide is known to act directly through the opioid receptors (μ-1 opioid receptor, in particular), and has been shown to be the major active metabolite of morphine. As such, in one embodiment, the composition herein comprises one or more therapeutically active doses of morphine-6 glucuronide in an excipient, for example, propylene glycol.

Morphine-6-glucuronide has a higher potency than morphine. (Wittwer and Kern, 2006). Morphine-6 glucuronide has a blood-effect site equilibration half-life of about 4 to 8 hours in a subject, and allows for greater control over the analgesic effect, as compared to codeine and most other opioids.

For purposes herein, a therapeutically active dose or amount of a morphine-6-X compound means an amount sufficient, such that the subject taking the compound experiences the intended medicinal effects, i.e., pain relief, anxiety relief, sense of well-being, etc. In the patient who demonstrates physical chemical and/or behavioral dependency, the dose of the morphine-6-X compound is sufficient to prevent withdrawal, anxiety and promote a sense of well-being and can be determined by the physician. A therapeutically active dose or amount is administered via a novel heat-activated drug inhalation device described herein, and can include from 0.01 to 15 mg of targeted opioid in the composition, as loaded in the novel heat-activated drug inhalation device.

In some embodiments, the dose for a target subject is determined via titration by the physician, where the dose is titrated until the intended medicinal effect is achieved for that particular target subject. For example, based on a variety of differing physiologic parameters, the dose that is active for one patient is typically not active for another patient. As such and for example, a patient practicing the embodiments herein, may titrate a heat-activated morphine-6-X compound composition to establish a proper therapeutically active dose for him or herself, under the supervision of the attending physician and with restrictions, such as a maximum dose, limits on oxygen saturation, and dosing frequency.

In addition, a patient could titrate, under a physician's supervision, any of the targeted opioids to obtain the therapeutically active dose for the particular compound, i.e., morphine, 6 monoacetylmorphine, synthetic opioids, morphine-6-X compounds, and the like, under the supervision of the attending physician and with restrictions, such as a maximum dose, limits on oxygen saturation and dosing frequency. In one embodiment, the subject would start with a composition having 0.5 mg of targeted opioid. If the physiologic parameter was not met within a predetermined time frame, the subject would titrate another composition having 0.5 mg of targeted opioid, as pre-determined by the attending physician and meeting the oxygen saturation minimum level. The process would continue until the physiologic parameter has been met, or until the limits of dose or until the oxygen saturation is unacceptable. Other starting doses are contemplated herein, for example, 0.1 mg, 0.2 mg, 1 mg, 2 mg, 5 mg. If the analgesic effect or other outcome target is not met within the parameters allowed, the patient should consult with the physician. The physician may now increase the total number of milligrams of targeted opioid as the active dose for the patient's administered dose.

The dose can be administered over the course of the novel heat-activated drug inhalation device being activated and heated one or more times, as prescribed by the physician or other health care professional. The timing of any one activation (inhalation) of the device for a dose event of targeted opioid can be regulated with a timer integrated within the medical device. For example, the timer may be set to as low as 1 second between activations, or up to 4, 6, 8, etc. hours. In general, administration of a targeted opioid should be over a shorter time frame for each dosing event, but under some circumstances will require the longer times. In typical embodiments, one dose event of a targeted opioid is administered via multiple inhalations over the course of the device being activated for about 30 to 90 seconds.

It is also noted that a novel heat-activated drug inhalation device can be loaded with one or more therapeutically active doses or concentrations from a targeted opioid composition. For example, the medical device described herein can be loaded with two or more active doses which consist of two or more ampoules. Once one ampoule has been emptied, the medical device will dispense future doses from an alternate full ampoule.

Manufacture and production of morphine-6 glucuronide has been previously described using a number of different protocols. For example, morphine-6-glucuronide can be produced using: (1) the imidate method (Fischer et al., J. Org. Chem. 1984, 49, 4988); (2) synthesis from morphine using alkali metal salts (WO 93/05057); (3) synthesis by selective enzyme-catalyzed hydrolysis of morphine-3,6-diglucoronide (Brown et al., Tetrahedron Letters, 1995, 36, 1117-1120); (4) using the Knorr Synthesis (Yoshimura et al., Chem Pharm Bull., 1968, 16, 2114-2119, and Lacy et al., Tetrahedron Letters, 1995, 36, 22, 3939-3950); (5) using a synthetic method from 3-O-pivaloyloxymorphine (U.S. Pat. No. 6,566,510); and (6) as a crystalline compound from water (U.S. Pat. No. 6,172,206). Each of these references are incorporated in their entirety for all purposes. Also, these are only illustrative of various manufacturing methods, other methods for morphine-6-glucuronide production are within the scope of the present disclosure.

The methods of action for morphine-6 bromide is similar to morphine-6 glucuronide. Morphine-6 glucuronide bromide shows good thermal stability as well as hydrolytic stability. Studies have shown that this bromide salt can be stored up to 6 years at room temperature with little or no degradation. Manufacture of morphine-6 glucuronide bromide has been previously described in WO 2004016633, which is incorporated by reference for all purposes.

Morphine-6-sulfate (and its analogs) also act in a similar manner as morphine-6-glucuronide Like morphine-6-glucuronide and its bromide salt, morphine-6-glucuronide sulfate shows a relatively long duration of activity and highly useful stability. Manufacture of morphine-6-glucuronide sulfate and its activity are described in U.S. Pat. No. 6,403,602, which is incorporated by reference for all uses.

Morphine-6-acetate also acts in a similar matter as morphine-6-glucuronide. As above, morphine-6-glucuronide acetate shows relatively long duration of activity and enhanced stability. Synthesis of morphine-6-glucuronide acetate is described in either Varadi, Andras et al., Eur J of Phar Sci, 42(1-2), 65-72; 2011 or Barrett et al., J or Liq Chrom., 17(17), 3727-33; 1994, each of which is incorporated by reference for all uses.

As noted above, targeted opioids can be administered alone or in combination with an excipient. However, temperatures required for heat-activation are much higher for the solid opioids. The risk for decomposition of the targeted opioid is substantially higher at these higher temperatures. Excipients, as discussed above, refer to safe and acceptable substances that can be combined with targeted opioids during the heat-activation process which lower the heat required to activate the targeted opioid as well as reduce the energy requirements to meet the high temperatures. Combination of the targeted opioid and one or more excipients can occur in the medical inhalation device itself, or can be completed before either is loaded into the medical inhalation device.

In one embodiment, the excipient is propylene glycol. Propylene glycol is an organic compound with the formula C₃H₈O₂. In various embodiments, the targeted opioid can be approximately 0.5% or more of the composition, while the propylene glycol makes up the remainder. Unless otherwise noted with respect to compositions herein, a percent of a targeted opioid or excipient or other material refers to a weight percent. In other embodiments, the targeted opioid can make up from approximately 1% to 99% of the composition, or 1% to 75%, 1% to 50%, 1% to 25%, 1% to 20%, or 1% to 10% of the composition, with the propylene glycol making up the remainder. In other embodiments, the composition includes a mixture of all known ratios of various excipients, for example PG/VG, and any known flavorants that are deemed to be GRAS. In some cases, some amount of water or alcohol may be combined with the targeted opioid and excipient. In other embodiments, compounds to provide an appropriate pH, compounds to extend stability of the active ingredient(s), or compounds such as flavor enhancing compounds may be included to improve taste and smell of the compositions.

In another embodiment, the excipient is glycerin, vegetable glycerin for example. In various embodiments, the targeted opioid can be approximately 0.5% or more of the composition, while the glycerin makes up the remainder. In other embodiemnts the composition includes a mixture of all known ratios of PG/VG and any known flavorants that are deemed to be GRAS In still other embodiments, the targeted opioid can make up from approximately 1% to 99% of the composition, or 1% to 75%, 1% to 50%, 1% to 25%, 1% to 20%, or 1% to 10% of the composition, with the glycerin making up the remainder. In some cases, some amount of water or alcohol may be combined with the targeted opioid and glycerin.

In yet another embodiment, the excipient is polysorbate, polysorbate 40 for example. In various embodiments, the targeted opioid can be approximately 0.5% or more of the composition, while the polysorbate makes up the remainder. In other embodiments, the targeted opioid can make up from approximately 1% to 99% of the composition, or 1% to 75%, 1% to 50%, 1% to 25%, 1% to 20%, or 1% to 10% of the composition, with the polysorbate making up the remainder. In some cases, some amount of water or alcohol may be combined with the targeted opioid and polysorbate.

Compositions may include only one type of excipient, or may include a combination of excipients. For example, a composition may include a targeted opioid, amorphine-6-glucuronide compound for example, propylene glycol and polysorbate, or a targeted opioid, glycerin and polysorbate. The compositions may also include more than one targeted opioid in combination with the one or more excipients. For example, a composition may include morphine-6-glucuronide, morphine-6-bromide, and glycerin.

Where an opioid composition includes alcohol, it may be present from about 0.05% to 40%, and more typically, 0.1% to 30%, of the excipient. In other embodiments the opioid composition includes from about 2% to 20% alcohol of the excipient.

Where an opioid composition includes water, it may be present from about 0.05% to 40%, and more typically, 0.1% to 30%, of the excipient. In other embodiments the opioid composition includes from about 2% to 20% water of the excipient.

In some embodiments, the opioid compositions include a combination of alcohol and water, and the combination may be present from about 0.05% to 40%, and more typically, 0.1% to 30%, of the excipient. In other embodiments the opioid composition includes from about 2% to 20% water and alcohol as compared to the excipient.

Beyond solubility, the excipients herein lower the temperature at which phase change to a gaseous state occurs for the targeted opioid composition. In some embodiments, the heating element in the device contacts the targeted opioid, a morphine-6-X compound for example, and excipient solution to cause activation, i.e., where the solution changes from a liquid state to a gaseous state, thermal vaporization. For purposes herein the heating element will heat the targeted opioid solution to a temperature of about 50° C. to about 500° C., and any temperature there between. In some embodiments, the heating element will heat the solution to a temperature of 50° to 260° C., and any temperature there between, and more typically, from 170° C. to 240° C., and any temperature there between. In other embodiments, the heating element will heat the solution to a temperature of about 170° C. to about 200° C., and any temperature there between. Finally, in one embodiment, the heating element will heat the opioid composition to a temperature of about 200° C.

A targeted opioid composition for use in the novel heat-activated drug inhalation device is formulated such that heating of the opioid composition allows for delivery of a precise dose of the targeted opioid to a subject in a specified period of time. The targeted opioid composition is activated and inhaled by the subject through the novel heat-activated drug inhalation device.

Embodiments herein also include kits for the administration of a targeted opioid to a subject. A kit can include one or more therapeutically active doses of a targeted opioid composition, and a novel heat-activated drug inhalation device configured to administer the targeted opioid composition.

For purposes of the kit including the novel heat-activated drug inhalation device, a targeted opioid is combined with an excipient to provide an opioid composition having one or more therapeutic doses. In one aspect, the kit includes a specified number of single dose packaged targeted opioid compositions. A pharmacist would likely prepare the doses and provide the kit to the subject. A subject would load a new targeted opioid dose to the novel heat-activated drug inhalation device for each desired use. In other aspects, the kit would include a single volume of a targeted opioid composition, morphine-6-glucuronide and polysorbate 40 for example, to be loaded into the device, for example, a packaging of 3, 5, 10, 20, 30 or more doses for loading into the device at one time. In such cases, the packaged targeted opioid would be a cartridge for loading into the novel heat-activated drug inhalation device. However, the kit would likely be provided by a pharmacist or other like professional in a loaded condition such that the subject does not have access to unloaded composition, to limit the potential of abuse or non-precise loading. In such cases, the pharmacist would pre-load the device within the kit with a predetermined number of targeted opioid composition doses. The novel heat-activated drug inhalation device would be locked such that tampering with the device would cause destruction of the targeted opioid. In yet still another embodiment, the pharmacist would dispense a cartridge or a package of cartridges that have been premixed by the manufacturer at differing concentrations, which could include the active pharmaceutical ingredient, the excipient, and flavoring-called somnivape™(™ Pending), which is activated only by heating and inhaling.

Kits including the novel heat-activated drug inhalation devices are shown in related application entitled “Method, System and Apparatus for Controlled Delivery of Opioid and other Medications,” by the same inventor herein, which is incorporated by reference for all purposes. Novel heat-activated drug inhalation devices are typically hand held and portable. The device may have a shell, a mouthpiece, an air inlet, an atomizer, one or more storage compartments, one or more pumps, a pressure sensor, a heater, a heat sensor, a battery, heat and pressure control, and optionally an on/off regulator tied to a pulse oximeter, a timer, a regulator which can vary and limit the dosing, a tamper resistant feature which destroys the ampoule to prevent abuse, and finger print scanner (or other biometric or locking device). In some embodiments, a soda lime trap can capture and inactivate un-inhaled drug prior to exhalation into the atmosphere. In more typical versions of the novel heat-activated drug inhalation devices herein, the device has at least a shell, mouthpiece, air inlet, targeted opioid compartment, a battery, and a heater. The novel heat-activated drug inhalation device for the kits herein can be disposable or reusable.

Kit embodiments will also include a set of instructions on the use and operation of the novel heat-activated drug inhalation device, as required and approved by regulatory agencies, including instructions on how to set the finger print input and pulse oximeter data. Instructions can include information related to one of the targeted opioids dose instructions, side effects, alternatives, and the like. A separate set of information would be prepared for each targeted opioid: morphine, morphine sulfate, 6 mono acetyl morphine, and the morphine-6-X compounds. In some embodiments, a smartphone application, with blue tooth communication, can give patients instructions, prompts for dosing, timing and other valuable information that relates to whatever particular disease state they are being treated for.

Kit embodiments can/may also include a pulse oximeter, or other like device. Typically, the pulse oximeter would be configured to be in communication with the novel heat-activated drug inhalation device, such that non-conforming blood oxygen levels in the patient would keep the device in the off or non-operational state.

Kit embodiments may further include one or more doses of naloxone hydrochloride. Naloxone hydrochloride is a medication for blocking the effects of opioid overdose. The naloxone hydrochloride can be prepared for delivery via the novel heat-activated drug inhalation device, or can be provided as a pill (orally administered), nasal spray (via the nasal passage) or injectable (intravenous or intramuscular). Kits may also include multiple forms of the naloxone hydrochloride, as the drug's effects have different modes of timing. For example, a kit can have one or more naloxone hydrochloride pills and one or more intra muscular (IM) naloxone hydrochloride doses for injection. Where the naloxone hydrochloride is provided as an injectable, the kit may also include one or more sterile syringes for administration of the drug. In some cases, the naloxone hydrochloride comes loaded in a syringe prepared for a single dose administration. Inclusion of naloxone hydrochloride is particularly useful where the active ingredient in the kit is morphine or 6-mono-acetyl-morphine. In some embodiments, an accelerometer is included to detect motion. If motion goes undetected for a period of time a GPS sensor may then notify EMS of a medical emergency and the patients location.

Embodiments herein also include methods for administering a targeted opioid composition, for example, morphine-6-glucuronide, to a patient. When using other opioids such as morphine or fentanyl or any other non-morphine-6 derivative potent analgesic, an initial analysis of the subject's health is necessary, or at the very least, an analysis of the subject's blood oxygen level. Because opioids other than the morphine-6-glucuronide compounds depress the user's respiratory function, it is important that the subject have tested an appropriate blood oxygen level prior to administration of opioids, other than morphine-6-glucuronide. As shown in Table 1, the subject's blood oxygen level results in a number of possible results. Other health measures may also be utilized in combination (or replacement) with the blood oxygen level of the subject, for example, the subject's pulse rate, blood pressure, respiratory rate, perspiration, GPS, activity (accelerometer) and the like. The following steps list the basic steps in the method described herein:
1. Activate and unlock the device with a biometric sensor, passcode or other means.
2. Measure patient oxygen saturation.
(If acceptable, go to step 3. If unacceptable, refer to Table 1.)
3. Heat pre-specified volume of opioid composition to the point of phase change to a gaseous state.
4. Patient inhales the heat-activated opioid composition.

First, the device is activated and unlocked and the oxygen saturation of the patient measured. Where a subject has an appropriate blood oxygen level, administration of the opioid may proceed. The subject accesses the novel heat-activated drug inhalation device and signals the device to heat a therapeutically active dose of the opioid composition. The opioid composition and strength of the composition would have been predetermined and loaded into the device. Heat-activation would proceed at a temperature of between 50° to 500° C., and more typically, 50° C. and 260° C. The heat-activated morphine-6-glucuronide, for example, is administered through the device to the subject via inhalation. In typical methods, the subject will inhale the heat-activated composition over a period of 30 to 90 seconds, using multiple inhalations. The timing of inhalations and dosing events may proceed under any number of different manners.

TABLE 1
Rules for Oxygen Saturation

| Lower Limit | Upper Limit | | | |
|---|---|---|---|---|
| for Oxygen | for Oxygen | | | |
| Saturation | Saturation | Rule | Device Display | Device Action |
| 94% | 100% | Success - an | Okay to administer | Subject can access |
| | | appropriate blood | opioid | device |
| | | oxygen level | | |
| 90% | <94% | Subject instructed | Try again, but after | Device will |
| | | to take deep | 3 attempts, device | become locked for |
| | | breaths | locked and patient | predetermined |
| | | | instructed to visit | amount of time; |
| | | | physician | once time has |
| | | | | passed the device |
| | | | | will unlock and |
| | | | | patient can try |
| | | | | again |
| <90% | | Subject instructed | Try again, may | Seek immediate |
| | | to take deep | result in | medical attention, |
| | | breaths | emergency call if | and/or use |
| | | | blood oxygen level | enclosed naloxone |
| | | | remains below | hydrochloride |
| | | | 90%; patient may | |
| | | | also be instructed | |
| | | | to administer | |
| | | | naloxone | |
| | | | hydrochloride | |

Another method for administering a morphine-6-X compound composition, or other opioid composition described herein, to a subject is described herein. A subject in need of a morphine-6-X compound administration would initially be required to match his or her finger print (or locking device combination) with the stored finger print data on the device. A matched score would allow access to the device, and an unmatched score would result in temporary shut-down of the device. Once a matched finger print score is obtained, the subject would provide health statistics, like blood oxygen levels, to allow the device to continue. As above, a blood oxygen level at or above 94% would provide assurances that administration of the morphine-6-X compound to the subject would not result in respiratory failure. Non-conforming blood oxygen levels would result in temporary shutdown of the device, including potential instructions to the user to take naloxone hydrochloride, or alert emergency services. In such instances, a GPS could be included in the device, or be part of the kit, to notify emergency responders of the location of the subject. In yet other embodiments, a carbon dioxide analysis may be included with similar critical carbon dioxide levels that would inhibit use of the device, or if low enough, trigger a GPS signal to emergency responders. In yet another embodiment of the device, a flow meter can be utilized to assess adequate inhalation and or timing and delivery of the inhale dosage.

A subject that has access to the novel heat-activated drug inhalation device can now activate the device for a dose of a morphine-6-X compound composition, for example. However, in this method embodiment, the device tracks the number of morphine-6-X compound doses as administered over the past 24 hours, and compares that number with the predetermined amount allowed for that subject. The method also allows for a shorter time comparison as well, for example, a comparison of the number of administered doses over the past 2 hours, compared with the allowed dosage for the same amount of time. Any number of different comparisons and calculations can be used to determine whether the subject can receive another dose of the active ingredient. If the comparison allows for activation and administration of a composition dose, the device heats the appropriate active composition and the subject inhales the heat-activated composition over a period of time. Where the determination is that the subject is not entitled to another dose of active ingredient, the device will not administer a dose. In some method embodiments, the device could signal the subject when a next dose of active ingredient is available under the subject's predetermined dosing schedule. Signals may include LED lights, sounds, messaging to a cell phone, and the like. In some cases, the subject's health care provider would also receive notice that the subject is due for another dose administration. The following steps list the basic steps in the method described herein:
1. Activate and unlock the device with a biometric sensor, passcode or other means.
(If the biometric data or passcode do not match, the device cannot be used. If the biometric data or passcode match, the device unlocks, go to step 2)
2. Measure patient oxygen saturation.
(If acceptable, go to step 3. If unacceptable, refer to Table 1.)
3. Patient provides health data.
4. If time for dose, activate the heating component of the device.
5. Heat pre-specified volume of opioid composition to the point of phase change to a gaseous state.
6. Patient inhales the heat-activated opioid composition.

Method embodiments herein also include the administration of naloxone or naltrexone to the subject, where necessary. The naloxone or naltrexone may be administered with the targeted opioid, or upon an indication that the subject is undergoing some level of opioid dependence or overdose. Access to naloxone can be automatic, i.e., the device upon measuring a low blood oxygen level, automatically unlocks and loads a dose of naloxone, or the subject can access a naloxone pill or injectable.

Methods herein are useful for subjects in need of severe pain and anxiety relief. This is particularly true for patients overcoming cancer pain, chronic benign pain, surgery, and opioid addiction. In some cases, the patient is experiencing a physically chemical or behavioral opioid dependence in need of one or more maintenance doses of a targeted opioid, for example, morphine-6 glucuronide.

Finally, neonatal abstinence syndrome is an ongoing medical problem for patients who are born addicted to opioids. The current available treatment options are inadequate and fraught with difficulties. Neonatal Abstinence Syndrome, Karen McQueen, R.N., Ph.D., and Jodie Murphy-Oikonen, M.S.W., Ph.D. N Engl J Med 2016; 375:2468-2479, Dec. 22, 2016 DOI: 10.1056/NEJMra1600879).

The cost of treating newborns born with neonatal abstinence syndrome is currently in the tens of thousand dollars per patient. Morphine-6-X compounds are unique in that they do not cause physical withdrawal. Newborn babies can be put in an inhalation tent whereby they would inhale a morphine-6-X compound composition as described herein, combined with any combination of excipients. In some embodiments, a combination of active and excipient (PG/VG) and a pleasant flavorant are combined. In such embodiments, full opioid withdrawal could safely occur within 72 hours. The infants should then be able to be discharged without any opioids from the hospital.

All references cited herein are incorporated in their entirety.

What is claimed is:

1. A composition, comprising:
a therapeutically active dose of a morphine-6-X compound; and
an excipient, wherein
the morphine-6-X compound is fully soluble in the excipient; and
the therapeutically active dose is for delivery by inhalation.

2. The composition of claim 1, wherein the morphine-6-X compound is morphine-6-glucuronide or morphine-6-bromide.

3. The composition of claim 1, wherein the morphine-6-X compound is morphine-6-sulfate or morphine-6-acetate.

4. The composition of claim 1, wherein the excipient is propylene glycol.

5. The composition of claim 1, wherein the excipient is glycerin.

6. A kit comprising:
one or more therapeutically active doses of a targeted opioid;
a placebo solution of an excipient; and
a novel heat-activated drug inhalation device configured to administer either the one or more therapeutically active doses of the targeted opioid or the placebo solution.

7. The kit of claim 6, further comprising instructions on how to load the device with both the one or more therapeutically active doses of targeted opioid and the placebo solution.

8. The kit of claim 6, wherein the one or more therapeutically active doses of targeted opioid is one or more therapeutically active doses of a morphine-6-X compound.

9. The kit of claim 6, further comprising one or more doses of naltrexone hydrochloride.

10. The kit of claim 9, further comprising sterile syringes or sterile nasal spray dispensing devices for administration of the naloxone hydrochloride.

11. A method for administering a morphine-6-X compound to a subject, comprising:
testing the subject to confirm an acceptable blood oxygen level;
heating a composition of a morphine-6-X compound at a temperature of from 160° C. to 260° C. in an appropriate device; and
administering the heat-activated morphine-6-X compound through the device to the subject.

12. The method of claim 11, further comprising:
confirming the subject's identity prior to testing the subject for acceptable blood oxygen levels.

13. The method of claim 12, wherein the acceptable blood oxygen level is above 94%.

14. The method of claim 12, wherein the confirming the patient's identity is by biometric analysis.

15. The method of claim 11, wherein the composition of morphine-6-X compound consists essentially of a therapeutic dose of morphine-6-glucuronide and propylene glycol.

16. The method of claim 11, wherein testing the subject to confirm an acceptable blood oxygen level is through pulse oximetry technology.

17. The method of claim 11, further comprising:
regulating the number of times the subject can receive doses of the morphine-6-X compound in a predefined period of time.

18. The method of claim 11, wherein the morphine-6-X compound is morphine-6-glucuronide.

19. The method of claim 11, wherein the morphine-6-X compound is morphine-6-bromide.

20. The method of claim 11, wherein the morphine-6-X compound is morphine-6-sulfate.

21. The method of claim 11, wherein the morphine-6-X compound is morphine-6-PO₄.

22. A composition, comprising:
a therapeutically active dose of a target opioid; and
an excipient, wherein
the target opioid is fully soluble in the excipient; and
the therapeutically active dose is for delivery by thermal vaporization.

23. The composition of claim 22, wherein the target opioid is morphine.

24. The composition of claim 22, wherein the target opioid is a synthetic opioid.

25. The composition of claim 22, wherein the target opioid is a semi-synthetic opioid.

26. The composition of claim 22, wherein the excipient comprises a combination of glycerin and propylene glycol.

27. The composition of claim 22, wherein the excipient comprises glycerin, propylene glycol and polysorbate.
